(19) United States
(12) Patent Application Publication
(10) Pub. No.: US 2016/0052902 A1
(43) Pub. Date: Feb. 25, 2016

(54) CATALYTIC SYNTHESIS OF REDUCED FURAN DERIVATIVES

(71) Applicant: ARCHER DANIELS MIDLAND COMPANY, Decatur, IL (US)

(72) Inventors: Alexandra Sanborn, Lincoln, IL (US); Thomas P. Binder, Decatur, IL (US)

(21) Appl. No.: 14/781,524

(22) PCT Filed: Mar. 27, 2014
(86) PCT No.: PCT/US2014/031933

§ 371 Date: Sep. 30, 2015

Related U.S. Application Data
(60) Provisional application No. 61/815,775, filed on Apr. 25, 2013.

(51) Int. Cl. C07D 307/42

(52) U.S. Cl. CPC C07D 307/42

(57) ABSTRACT

The present invention relates to catalytic synthesis of furan derivatives from alkoxymethylfurfural ethers or acyloxymethylfurfural esters. More particularly, the invention pertains to furan derivatives obtained by use of a multifunctional catalyst system to carry out both hydrogenation of furan starting material and hydrolysis of the reduced furan derivative in a single reaction. The process allows recovering and recycling of alcohol or acid from the reaction product.

### FIELD OF THE INVENTION

The present invention relates to catalytic synthesis of furan derivatives. More particularly, the invention pertains to furan derivatives obtained by use of a multifunctional catalyst system in controlled hydrogenation of alkoxymethylfurfural ethers or acyloxymethylfurfural esters to produce furan derivatives.

### BACKGROUND OF THE INVENTION

Biomass contains carbohydrates (hexoses and pentoses) that can be converted into industrial chemicals from renewable hydrocarbon sources. The production of furan derivatives from biomass sugars promotes achieving sustainable energy supply and chemicals production.

Agricultural raw materials such as starch, cellulose, sucrose or inulin are inexpensive starting materials for the manufacture of hexoses, such as glucose and fructose. The dehydration of fructose produces 2-hydroxymethyl-5-furfuraldehyde, also known as hydroxymethylfurfural which is abbreviated, HMF. The structure of HMF is shown below:

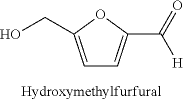

HMF represents one key intermediate substance readily derived from renewable carbohydrates. One of the concerns with HMF is that it has limited uses as a chemical per se, other than as a source for making derivatives. Furthermore, HMF is rather unstable and tends to polymerize and/or oxidize with prolonged storage.

Efforts to deal with HMF's instability and tendency to polymerize have sought to either form more stable and easily separated HMF derivatives, for example, HMF esters and ethers. Esters and ethers of hydroxymethylfurfural can be converted to various furan derivatives by hydrogenation.

### BRIEF SUMMARY OF THE INVENTION

The following presents a simplified summary of the invention in order to provide a basic understanding of some of its aspects. This summary is not an extensive overview of the invention and is intended neither to identify key or critical elements of the invention nor to delineate its scope. The sole purpose of this summary is to present some concepts of the invention in a simplified form as a prelude to the more detailed description that is presented later.

The present invention provides multifunctional catalyst systems and methods to convert esters or ethers of alkoxymethylfurfural to various furan derivatives in a controlled manner. The method involves hydrogenating a starting material containing at least one of an alkoxymethylfurfural ether or ester with hydrogen in the presence of a catalytic system under mild conditions to produce a reduced furan derivative. As used herein, mild conditions means an operational temperature of less than 150° C., or more typically less than 100° C., and/or at a pressure of less than 1250 psi (86 bar). The method further involves hydrolyzing at least one of an ether or an ester bond, respectively, from the alkoxymethylfurfural ether or acyloxymethylfurfural ester, and recovering at least one of an acid or alcohol and a reduced furan derivative.

FIG. 1 represents a general schematic of a series of inter-related hydrogenation reaction pathways for converting alkoxymethylfurfural esters or ethers to produce a variety of furan derivatives therefrom. One desirable derivative of HMF ethers is a partially reduced furan derivative in which the aldehyde moiety of HMF is converted to an alcohol. Furan derivatives produced after hydrolyzing at least one of an ether or an ester bond could then be used as chemicals or fuels, depending on the remaining functionality.

A feature of the present method is that it enables direct synthesis of reduced furan derivatives from alkoxymethylfurfural esters or ethers, using a multifunctional catalyst system. A multifunctional catalyst system comprises one or more catalysts having a plurality of functionalities and may include a promoter. A particular advantage of this invention is that the alcohol or acid produced can be recovered and recycled. Recycling the recovered alcohol or acid comprises using the alcohol in a second reaction operable to form an alkoxymethylfurfural ether, or using the acid in a second reaction operable to form an acyloxymethylfurfural ester.

The use of a multifunctional catalyst system to carry out both hydrogenation of furan starting material and hydrolysis of the reduced furan derivative in a single reaction has not been reported. In addition, recovering and recycling of alcohol or acid from the reaction product has not been an option until now.

### BRIEF DESCRIPTION OF THE FIGURES

FIG. 1 is a general schematic process illustrating various hydrogenation pathways of acyloxymethylfurfural and alkoxymethylfurfural derivatives.

FIG. 2 is a pathway of an acyloxymethylfurfural or alkoxymethylfurfural ether to 2,5-dimethyltetrahydrofuran with the generation of an alcohol or acid.

FIG. 3 depicts the molar yield differences from hydrogenation of 5-butoxymethylfurfural at three different reaction times.

FIG. 4 shows the effect of a catalyst system concentration on the molar yield of furan derivatives according to an embodiment of the present method.

FIG. 5 depicts results of inventive examples using 5-butoxymethylfurfural, showing the effect of promoter on the percent (%) area of products obtained in ethyl acetate at 65° C., 1 hour and 60 bar.

FIG. 6 depicts bar charts showing results of inventive examples of hydrogenation of 5-butoxymethylfurfural, showing the effect of processing at three different reaction times and with two different solvents. EtOH=ethanol, EtOAc=ethyl acetate.

FIG. 7 depicts the product distribution differences resulting from hydrogenation of 5-acetoxymethylfurfural with four different catalysts systems.

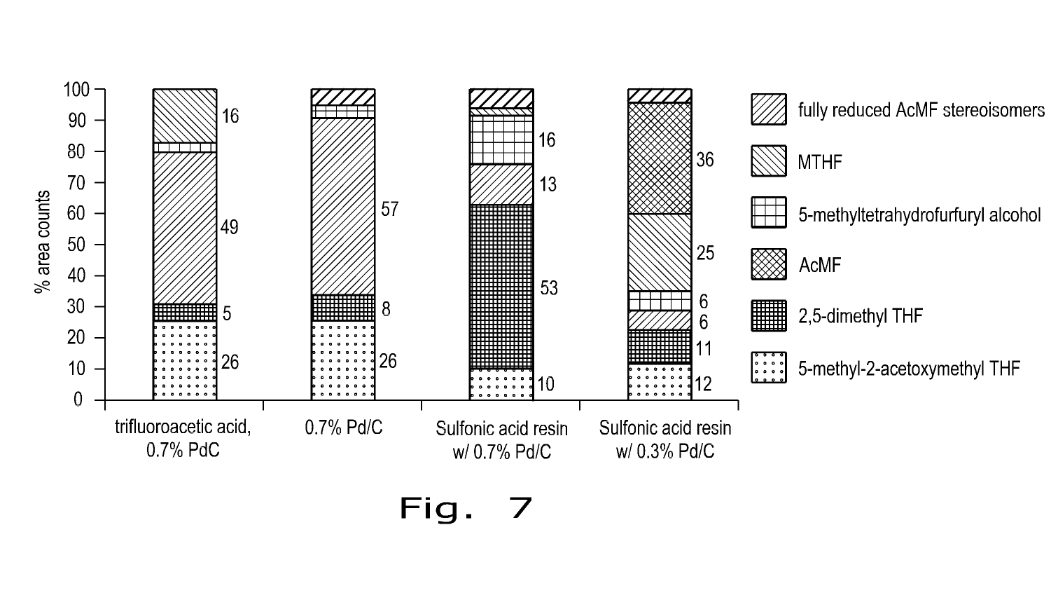

### DETAILED DESCRIPTION OF THE INVENTION

So there is no ambiguity, the term “hydroxymethylfurfural ether,” “alkoxymethylfurfural ether,” and “HMF ether” are used interchangeably herein and refer to molecules that are more technically designated R-5′ alkoxy methyl furfural ethers having the general structure:

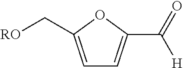

The terms “HMF ester,” “acyloxymethylfurfural ester,” “alkoxymethylfurfural ester,” and “hydroxymethylfurfural ester” are used interchangeably herein and refer to molecules that are more technically designated R-5′ acyl methyl furfural esters having the general structure:

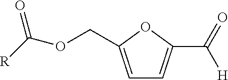

In each case R is an alkyl group that may be at least one of straight chained, cyclic or branched, having from 1 to 24 carbon atoms, and may also contain oxygen, nitrogen or sulfur. Some preferred alkyl groups are the C1 to C5 alkyl moieties such as methyl, ethyl, n-propyl, i-propyl, i-butyl, n-butyl, i-amyl and n-amyl. These alkyl substituted HMF compounds can be derived from natural bio-based sources. For example, methyl substituted HMF ethers can be synthesized from methanol derived from biomass gasification. Alternatively, the C1 to C5 alkyl groups can be obtained from ethanol and fusel oil alcohols. Fusel oil is a distillation by-product of fermentations to make ethanol whose main components are isopentyl alcohol and 2-methyl-1-butanol, and to a lesser degree contains isobutyl alcohol, n-propyl alcohol, and small amounts of other alcohols, esters and aldehydes. In addition, n-butanol may be derived from the fermentation to make acetone/ethanol or from the catalytic condensation of ethanol.

When sugars from biomass are subjected to acid-catalyzed dehydration, complicated product mixtures are obtained. When the acid-catalyzed dehydrations are performed in the presence of a solvent comprising an alcohol, alkoxymethylfurfural ethers are obtained in the product mixture, together with carbohydrates, alkyl levulinates, humins, and other poorly characterized products. This complicated product mixture containing alkoxymethylfurfural ether can be a furan starting material for the hydrogenation according to the methods herein. When the acid-catalyzed dehydrations are performed in the presence of solvent comprising an organic acid, acyloxymethylfurfural esters are obtained in the product mixture, together with carbohydrates, alkyl levulinates, humins, and other, poorly characterized products. This complicated product mixture containing acyloxymethylfurfural ester can be a furan starting material for the hydrogenation according to the methods herein. In the present disclosure we disclose a multifunctional catalyst system which, in operation, is able to selectively yield furan derivatives from complicated product mixtures containing at least one of alkoxymethylfurfural ether or acyloxymethylfurfural ester.

Suitable furan starting materials containing alkoxymethylfurfural ethers for the present disclosure may contain varying levels of alkoxymethylfurfural ethers. When the furan starting materials are subjected to hydrogenation using a multifunctional catalyst system of the present disclosure, the desired furan derivatives are obtained even in the presence of carbohydrates, alkyl levulinates, humins, and other, poorly characterized products in the furan starting material. In an embodiment, a furan starting material containing as little as 5% alkoxymethylfurfural ether on a dissolved solids basis in the form of butoxymethylfurfural can be converted to a reduced furan derivative in high yield.

Suitable furan starting materials containing acyloxymethylfurfural esters for the present disclosure may contain varying levels of acyloxymethylfurfural esters. When the furan starting materials are subjected to hydrogenation using a catalyst system of the present disclosure, the desired furan derivatives are obtained even in the presence of carbohydrates, alkyl levulinates, humins, and other poorly characterized products in the furan starting material. In an embodiment, a furan starting material containing as little as 5% acyloxymethylfurfural ester on a dissolved solids basis in the form of acetoxymethylfurfural can be converted to a reduced furan derivative in high yield.

The term “furan derivatives” may refer to a partially or a fully reduced alkyl furan including but not limited to: 2,5-dimethyl-tetrahydrofuran (DMTHF), (5-(butoxymethyl)furan-2-yl)methanol, 5-methyl-2-butoxymethylfuran, 5-methyl-2-butoxymethyltetrahydrofuran, 2,5-dimethylfuran, (5-(butoxymethyl)tetrahydrofuran-2-yl)methanol, 2-(butoxymethyl)-5-(ethoxymethyl)furan, 2-(butoxymethyl)-5-(ethoxymethyl)tetrahydrofuran, (5-methylfuran-2-yl)methanol, (5-methyltetrahydrofuran-2-yl)methanol, 2-(butoxymethyl)furan, 2-(butoxymethyl)tetrahydrofuran, 2-methylfuran, 2-methyltetrahydrofuran, (5-formylfuran-2-yl)methyl acetate, (5-methyltetrahydrofuran-2-yl)methyl acetate, (5-(hydroxymethyl)tetrahydrofuran-2-yl)methyl acetate, (tetrahydrofuran-2-yl)methyl acetate, (5-(hydroxymethyl)furan-2-yl)methyl acetate.

Starting with an acyloxymethylfurfural ester such as (5-formylfuran-2-yl)methyl acetate (A), the following are examples of some of the furan derivatives that can be made according to the present method: (5-(hydroxymethyl)furan-2-yl)methyl acetate (B) and (5-(hydroxymethyl)tetrahydrofuran-2-yl)methyl acetate (C), which respectively are partially and fully reduced, respectively. The by-products are (5-methyltetrahydrofuran-2-yl)methyl acetate (D) and (tetrahydrofuran-2-yl)methyl acetate (E).

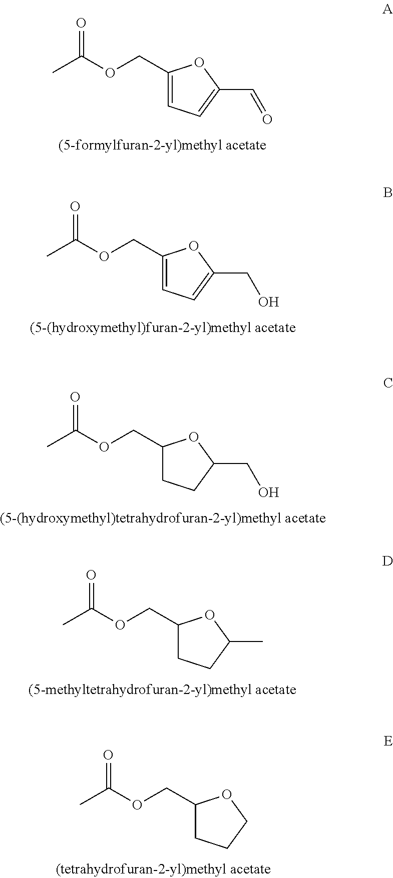

A feature of the present method is that it enables direct synthesis of furan derivatives from HMF esters or ethers, using a multifunctional catalyst system. As used herein, a “multifunctional catalyst system” is a combination of catalysts of different functionalities and may include a promoter. For example, a multifunctional catalyst system may include the use of catalysts, promoters or a combination thereof having several functionalities including but not limited to those of acid, base, hydrogenation, dehydration, ring opening, and combinations thereof with a furan starting material and conditions applied to generate reduced furan derivatives.

Another scope of the present invention is an ability to use a multifunctional catalyst system comprising a bifunctional catalyst or a combination of single catalysts having an acidic or an alkaline and hydrogenation functionality to synthesize furan derivatives from HMF esters or ethers. A bi- or multifunctionalized catalyst as used herein refers to catalysts that have two or more different functionalities and therefore are able to accelerate two or more different reactions, preferably the hydrogenation and the subsequent hydrolysis of the intermediate. This process may include the use of a multifunctional catalyst system having several functionalities including but not limited to those combining acid, base, hydrogenation, dehydration, ring opening and may include a promoter or combinations thereof.

The multifunctional catalyst system includes at least one of the following: a bifunctional catalyst; a combination of single catalysts having an alkaline and hydrogenation functionality; a catalyst having several functionalities, including acid, base, hydrogenation, dehydration, ring opening, and may include a promoter or combinations thereof. The multifunctional catalyst system may include at least a heterogeneous catalyst or a homogeneous catalyst. The multifunctional catalyst system exhibits degrees of selectivities for desired furan derivatives, such as: a) 2,5-dimethyl-tetrahydrofuran of at least about 30%; b) (5-(butoxymethyl)tetrahydrofuran-2-yl)methanol of at least about 80%; c) (5-(butoxymethyl)furan-2-yl)methanol of at least about 30%; d) 2-(butoxymethyl)-5-methyltetrahydrofuran of at least about 30%; e) (5-(hydroxymethyl)tetrahydrofuran-2-yl)methyl acetate of at least about 85%; f) 5-methyltetrahydrofuran of at least about 25%.

A catalyst for direct synthesis of reduced furan derivatives from HMF esters or ethers as used herein may include a transition metal of group VIII to XI. Some multifunctional catalyst systems that may be employed in the present process may include, for examples, Pd, Pt, Ru, or Ni, Cu—Cr, or different combinations of such metals. Platinum, palladium, rhodium, and ruthenium form highly active catalysts, which operate at lower temperatures and lower pressures of H2. Non-precious metal catalysts, especially those based on nickel (such as Raney nickel and Urushibara nickel) have also been developed as economical alternatives, but they are often slower or require higher temperatures. One exemplary catalyst comprises a palladium on carbon support (Pd/C) catalyst with about 0.5-10% palladium loading. The multifunctional catalyst system includes an alkaline promoter including but not limited to triethylamine, or an ion exchange resin of a polymer of unmodified 4-vinylpyridine residues and divinylbenzene residues.

A promoter as used herein refers to a substance which enhances the activity/and or the selectivity of the catalyst. In some embodiments the promoter can be a heterogeneous acid catalyst, which is a substrate comprising a solid material having an acidic group bound thereto. The solid material can be comprised of materials selected from acid clays, silicas, sulfated zirconia, molecular sieves, zeolites, ion exchange resins, heteropolyacids, carbon, tin oxide, niobia, titania and combinations thereof. In an embodiment, the substrate is a polymeric resin material such as polystyrene. The ion exchange resin may also be a sulfonated divinylbenzene/styrene copolymer resin. Some of these resin based catalysts are ordinarily used for cation exchange chromatography. Perhaps the most common acid group for cation exchange resins and other heterogeneous acid catalyst is a sulfonic group. Suitable heterogeneous acid catalysts containing a sulfonic group are Amberlyst™ and Amberlite™, Dianion™, and Lewatit™. Examples include Amberlyst™ 35, Amberlyst™ 15, Amberlyst™ 36, Amberlyst™ 70, XN1010, IRC76, and XE586 (Rohm & Haas), RCP21H (Mitsubishi Chemical Corp.), Dowex™ 50WX4 (Dow Chemical Co.), AG50W-X12 (Bio-Rad), and Lewatit™ S2328, Lewatit™ K2431, Lewatit™ S2568, Lewatit™ K2629 (Bayer Corporation), HPK25 (Mitsubishi), Nafion-50 (DuPont). Other acid groups bound to substrates may also be used as the promoter. Suitable examples of other promoters include CRP-200 phosphonic/polystyrene (Rohm & Haas).

In some embodiments the multifunctional catalyst system includes an acidic promoter that is at least one of a homogeneous acid, heterogeneous acid, a mineral acid, and/or an organic acid. The acidic promoter may comprise a homogeneous catalyst, such as a mineral acid. Suitable mineral acid catalysts include sulfuric acid, nitric acid, hydrochloric acid, phosphoric acid and the like. The acidic promoter may also comprise an organic acid including but not limited to p-toluenesulfonic acid, trifluoroacetic acid, levulinic acid, and p-methanesulfonic acid.

In an embodiment, the catalyst may contain a metal that is Pt, Pd, or Ni; however, Co, Cu, Ru, Re, Rh, Ir, Fe and/or combinations of the same, with or without a promoter, may also be employed. In some embodiments, the metal may be added to the reaction mixture for producing furan derivatives as a heterogeneous particulate powder. In more typical embodiments, the metal is bound to a substrate forming a heterogeneous metal catalyst substrate. Typical substrates include, but are not limited to kieselguhr, diatomaceous earth, silica and polymeric resin materials. One exemplary metal catalyst is represented by G-69B™, available from Sud-Chemie, (Louisville, Ky.) which is a powdered catalyst having an average particle size of 10-14 microns containing nominally 62% Nickel on kieselguhr, with a Zr promoter. Other suitable catalysts containing Ni include, but are not limited to, sponge nickel and G-96B™ also available from Sud-Chemie Corp. G-96B™ is a nickel on silica/alumina, 66% nickel by weight, particle size 6-8 microns. Another preferred nickel catalyst is G-49B™ available from Sud-Chemie Corp. Particle size is 7-11 microns and 55% nickel by weight. Another preferred catalyst is palladium on carbon, exemplified by the catalyst Pd/C. Another preferred catalyst is G22/2™ also available from Sud-Chemie Corp. G22/2™ is barium promoted copper chromite catalyst, 39% Cu and 24% Cr. Other suitable catalysts containing Cu include, but are not limited to, sponge copper available from Johnson Matthey. In yet another embodiment the catalyst can be a platinum catalyst, exemplified by the catalyst Pt/C. Carbon-supported, zirconia-supported or zeolite-supported metal catalysts are also envisioned as workable multifunctional catalyst systems with the present method.

In a preferred embodiment, the promoter and the hydrogenation catalyst are provided on the same substrate, forming a heterogeneous bifunctional catalyst. Exemplary catalysts of this nature include Amberlyst™ CH10 and CH28, each available from Rohm and Haas Company (Midland, Mich.). Amberlyst™ CH10 is a macroreticular palladium metal hydrogenation resin containing sulfonic acid as the acid promoter component. Amberlyst™ CH28 is a macroreticular styrene DVB copolymer palladium doped hydrogenation resin also containing sulfonic acid as the acid promoter component. Lewatit™ K7333 catalyst available from Lanxess (Germany) is a palladium-doped polymer based resin containing trialkyl ammonium groups in OH-form. The present invention utilizes these exemplary resins as multifunctional catalyst systems for an efficient one pot conversion of furan starting materials under mild conditions to produce a plurality of furan derivatives.

In general, the hydrogenation reactions of the present method can be performed under relatively mild conditions over a wide range of temperatures and pressures. As used herein, “mild conditions” refers to certain operational parameters within a reactor in which operational temperatures do not exceed about 150° C. and/or pressures do not exceed about 1,230 psi (˜85 bar). In a preferred practice, the temperature is usually 150° C. and the pressure is less than 1230 psi. The only upper limitation on pressure is what the reactor can bear so higher pressures can be used if desired. Typically, the reaction can be conducted at an operational temperature within a range from, for instance, ambient room temperature of about 18° C. to 20° C., up to about 130° C., and at a minimal pressure of about 85 psi (˜5.8 bar) up to about 1,015 psi (˜70 bar), or any combination of temperatures and pressures therein between. Desirably, the reactions have an operational temperature of less than or equal to 100° C., at a pressure of less than or equal to 950 psi (˜65.5 bar). We have discovered that by carrying out the hydrogenation under temperatures below 100° C., we are able to obtain furan derivatives of higher selectivity. Nonetheless, depending on a manufacturer's desired product mix, various permutations of temperature and pressure can be applied. For instance, in some embodiments, the operational temperature of the reaction is within a range from 25° C.-95° C. and the operational pressure is from about 90 psi-950 psi (e.g., about 88° C., and about 940 psi (˜65 bar)). In certain examples, one can hydrogenate and hydrolyze the alkoxymethylfurfural ether or ester at a temperature in a range from about 60° C. to about 98° C., and at a pressure in a range from about 290 psi (˜20 bar) to about 895 psi (˜62 bar) to obtain a furan derivative.

Alternatively, the hydrogenation reactions can be executed at an operational temperature in a range from about 20° C. to 88° C. or any combination therein. Particular examples may operate at temperatures in a range from about 40° C. to about 85° C. (e.g., about 50° C. or 55° C. to about 75° C. or 80° C.).

The operational pressure within the reactor is within a range from about 100 psi to about 940 psi. Particular examples may operate in between about 20 bar and about 895 psi (˜62 bar), typically in a range from about 300 psi (˜20.7 bar) or 320 psi (˜22.1 bar) to about 875 psi (˜60.3 bar) or about 880 psi (˜60.7 bar). Some embodiments may operate at pressures between about 500 psi (˜34.5 bar) or 725 psi (˜50 bar) and about 885 psi (˜61 bar).

Other temperatures may be within a range, for example, from about 35° C. to 92° C., and other operational pressures may be within a range, for example, from about 130 psi to 900 psi. The time can be suitably determined by taking into account the reaction conditions, the scale of the reaction, the multifunctional catalyst system, and the like. The hydrogenation reactions can be run for a predetermined time period, such as about 1 hour, up to about 5 or 6 hours or longer.

According to the present method, the reaction is performed at conditions that will allow one a degree of control in the reduction of the molecule, without having to heat the starting materials to high temperatures (i.e., >150° C.). Previously, hydrogenation reactions have been carried out under stringent conditions at high temperatures and pressures resulting in the formation of a plethora of products. As is apparent, an efficient, practicable process for selective hydrogenation, using a catalyst with sufficient activity and selectivity, would be a substantial contribution to the art.

In the catalytic synthesis of reduced furan derivatives, the selectivity can be changed in a controlled manner. As used herein, “controlled manner” means the adjustment of reaction conditions, such as temperature, pressure, time, and/or multifunctional catalyst system to achieve high selectivity of a reduced furan derivative from a furan starting material containing at least one of the alkoxymethylfurfural ether and acyloxymethylfurfural ester and simultaneously to suppress inter-related hydrogenation reaction pathways.

In various embodiments, the furan starting material comprises at least butoxymethylfurfural, and a portion of the starting material is converted to a reduced furan derivative.

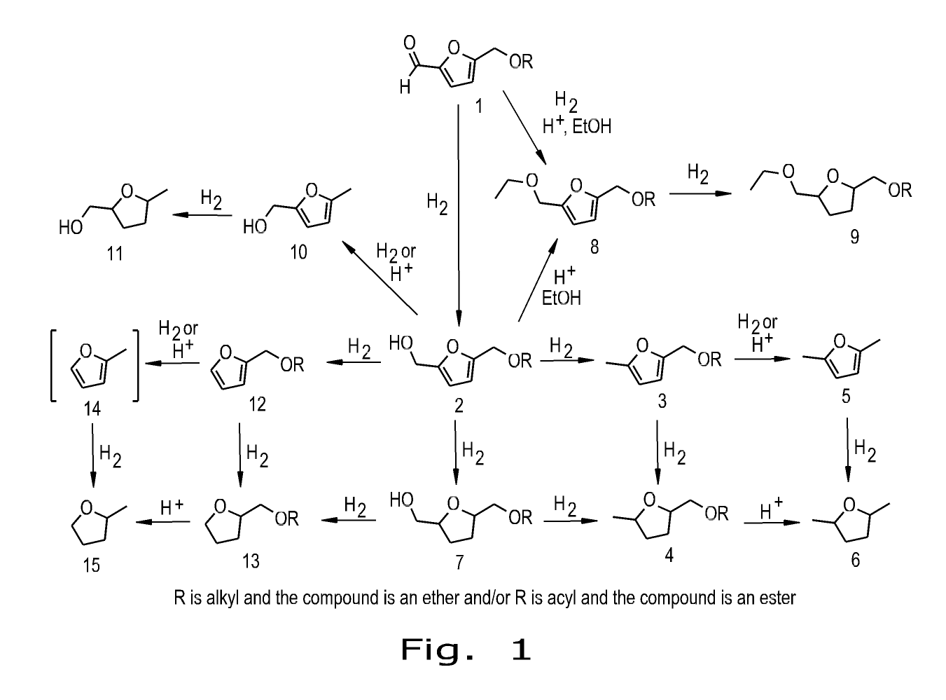

Returning now to FIG. 1, it is believed that during this reaction, BMF is partially reduced to form (5-(butoxymethyl)furan-2-yl)methanol (2) and/or fully reduced to form (5-(butoxymethyl)tetrahydrofuran-2-yl)methanol (7). Without being bound by theory, the plurality of reduced furan derivatives is believed to occur via one or more of the following pathways. In a first pathway, the partially reduced furan derivative 2 undergoes further hydrogenation to form the fully reduced furan derivative 7. In a second pathway, the partially reduced furan derivative 2 dehydrates leading to the formation of 5-methyl-2-butoxymethylfuran (3) and further hydrogenation of the furan ring leads to the formation of 5-methyl-2-butoxymethyltetrahydrofuran (4) or hydrolysis and/or hydrogenolysis of the ether bond generates butanol and 2,5-dimethylfuran (5) which is available for further hydrogenation to 2,5-dimethyltetrahydrofuran (6). In a third pathway, the partially reduced BMF derivative 2 in the presence of an alcohol solvent leads to the formation of the acetal of 1 via a carbocation intermediate which undergoes reduction to generate 8 and further reduction of the furan ring to form 9. The formation of 8 from 2 may also occur via an acid catalyzed pathway. In a fourth pathway, the partially reduced furan derivative 2 in the presence of an acid undergoes hydrolysis to generate butanol and (5-methylfuran-2-yl)methanol (10). Further hydrogenation of 10 leads to the formation of (5-methyltetrahydrofuran-2-yl)methanol (11). In a fifth pathway, decarbonylation of 2 and/or 7 leads to 2-butoxymethylfuran (12) and/or 2-butoxymethyltetrahydrofuran (13) which are available for further reaction generating butanol and 2-methylfuran (14) and/or 2-methyltetrahydrofuran (15). Effecting the reaction pursuant to the above described pathways generates butanol as a product, which is available for recycling in a second reaction operable to form at least one of a butoxymethylfurfural ether. One or more of the inter-related hydrogenation reaction pathways described above may occur simultaneously.

It has now been found that significant improvements in the selectivity to reduced furan derivatives can be obtained by controlled reaction conditions such as the addition of promoters in accordance with the present invention. Surprisingly, high selectivity for furan derivatives was obtained using a multifunctional catalyst system comprising a mixed catalyst, promoter or a combination thereof. Consequently, we were able to suppress inter-related hydrogenation reaction pathways, resulting in decreased formation of unwanted products.

The difference in conditions for obtaining at least one of the fully reduced furan derivative (7) and the reduced furan derivative 6 is the multifunctional catalyst system. Under an exemplary reaction at 25-60° C., 6 to 10 bar H2, with at least one of the alkoxymethylfurfural ether or acyloxymethylfurfural ester as the furan starting material, in the presence of a multifunctional catalyst system comprising an alkaline promoter and a palladium containing hydrogenation catalyst, the dominant product will be the fully reduced furan derivative 7 in 30 min to 2 hours of reaction time.

The choice of metal loading for each hydrogenation catalyst, with or without a promoter, may also control selectivity of reduced furan derivatives. In general, the metals may be present in various forms (e.g., elemental, metal oxide, metal hydroxides, metal ions etc.). Typically, the metal(s) at a surface of a support may constitute from about 0.25% to about 10% of the catalyst weight.

To make the reduced and hydrolyzed furan derivative, DMTHF (6), of at least 30% selectivity, the furan starting material containing at least one of the alkoxymethylfurfural ether or acyloxymethylfurfural ester is contacted with H2, and a multifunctional catalyst system comprising a homogeneous acidic promoter and a hydrogenation catalyst. A multifunctional catalyst system comprising a bifunctional catalyst and/or a combination of an heterogeneous acid catalyst promoter with a hydrogenation catalyst at a temperature of between 25 and 100° C. and a pressure of between about 6 to 60 bar for a time of between 1 to 2 hours produces DMTHF at least about 70% selectivity. In exemplary embodiments, the temperature is 60° C., the pressure is 60 bar, and the time is 1 hour.

The selectivity of the reaction can be controlled by loading of the multifunctional catalyst system, although longer reaction times will also enhance further hydrolysis and/or reduction. Using a multifunctional catalyst system comprising a bifunctional catalyst and loadings of 0.4 equivalents, the dominant product is the fully reduced furan derivative 7. The addition of higher equivalents of bifunctional resin catalyst system gave quantitative conversion of BMF, with higher yields of butanol and DMTHF. Similarly, increasing the reaction time can produce higher yields of butanol and DMTHF.

We are able to demonstrate considerable control of the selectivity of hydrogenation by varying the temperature, catalyst, and promoter. In the hydrogenation process, we are able to recover at least one of an alcohol or an acid, respectively, from at least one of the alkoxymethylfurfural ether or acyloxymethylfurfural ester, that permits one to produce selectively at least one of a partially or fully reduced furan derivative under relatively mild conditions of less than 100° C. and less than 950 psi. For instance, the method enables one to selectively reduce HMF ethers to produce furan derivatives including but not limited to substituted furan, substituted tetrahydrofuran or tetrahydrofuran compounds. Ring-opened products including hexanediols may also be formed.

The ability to simplify in one step the reduction and collection of the product can increase synthesis efficiency and save recovery costs. Another advantage of the present process is that a catalyst system can be separated from the reaction mixture in a simple manner and recycled to be optionally used again. For example, a palladium catalyst fixed on an ion-exchange resin is hardly deactivated because palladium metal is fixed on a matrix of the ion-exchange resin, and handling in recovery and reuse of such catalysts is extremely easy because the relative particle size of catalyst is very large.

In another aspect, we envision that an ability to convert an HMF ether or ester containing starting material to furan derivatives can be adopted to be processed by methods such as a continuous flow system, semi-batch reactions, or batch reactions according to embodiments of the present invention. For example, this reaction can be carried out batch wise or continuously in a fluidized bed, tubular reactor, column, or pipe. The process may involve the selective synthesis of a number of derivatives disclosed by simply changing the scale of the reaction, the reaction conditions, the catalyst system employed, and the like. Several reactors may be in line and the selectivity of product may be controlled by the switch of a valve or change in temperature, time or pressure. In short, the method described herein is not limited to batch reactions, but can be performed as a continuous process.

As various changes could be made in the above processes without departing from the scope of the invention, it is intended that all matter contained in the above description shall be interpreted as illustrative and not in a limiting sense.

### EXAMPLES

Table 1 provides a list of reference numbers, as used herein, for the various furan starting materials and furan derivatives in generic terms as shown in FIG. 1.

TABLE 1
Furan Starting Materials and Furan Derivatives
(R, R′ = acyl or alkyl)

| Ref. No. | Name |
|---|---|
| 1. | R-oxymethylfurfural |
| 2. | (5-(R-oxymethyl)furan-2-yl)methanol |
| 3. | 5-methyl-2-R-oxymethylfuran |
| 4. | 5-methyl-2-R-oxymethyltetrahydrofuran |
| 5. | 2,5-dimethylfuran |
| 6. | 2,5-dimethyltetrahydrofuran |
| 7. | (5-(R-oxymethyl)tetrahydrofuran-2-yl)methanol |
| 8. | 2-(R-oxymethyl)-5-(R′-oxymethyl)furan |
| 9. | 2-(R-oxymethyl)-5-(R′-oxymethyl)tetrahydrofuran |
| 10. | (5-methylfuran-2-yl)methanol |
| 11. | (5-methyltetrahydrofuran-2-yl)methanol |
| 12. | 2-(R-oxymethyl)furan |
| 13. | 2-(R-oxymethyl)tetrahydrofuran |
| 14. | 2-methylfuran |
| 15. | 2-methyltetrahydrofuran |

Although the general principles of the present disclosure are applicable to starting materials that have a variety of different acyl or alkyl R-substituents, the following discussion focuses for purposes of illustration on examples that have R=(a) n-butyl, or (b) acetyl groups. When dehydration reactions are carried in 1-butanol, the alcohol can react with 5-(hydroxymethyl)furfural (HMF) to yield a corresponding ether, 5-(butoxymethyl)furfural (BMF, 1a). In the same manner, the acyl ester, 5-acetoxymethylfurfural (AcMF, 1b), can be formed when acetic acid is used in the dehydration reaction.

Referring to FIG. 1, for example, in the hydrogenation of 5-(butoxymethyl)furfural (BMF, 1a) or 5-acetoxymethylfurfural (AcMF, 1b), a fully reduced furan derivative (7) species is formed with greater than 90% selectivity when the hydrogenation is conducted with a multifunctional catalyst system comprising palladium catalyst on carbon support (e.g., ≦5% Pd/C) in the presence of a alkaline resin (e.g., Purolite™ 5149 or Reilly™ 425). In the presence of a palladium-doped acid resin (e.g., Amberlyst™ 28), the hydrogenation yields predominantly dimethyl tetrahydrofuran (DMTHF, 6) with about 60% to about 70% selectivity.

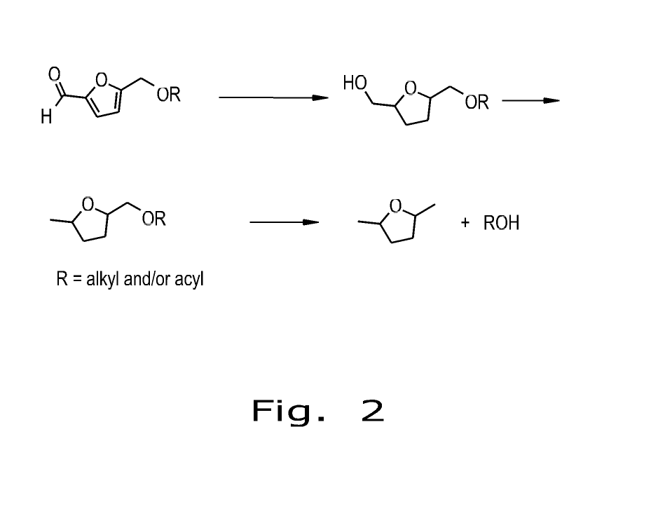

After hydrolyzing the ether or ester bond, one is able to recover the 1-butanol and acetic acid, respectively for recycling in a second reaction operable to form at least one of an alkoxymethylfurfural ether or an acyloxymethylfurfural ester (See e.g., U.S. Pat. No. 7,679,490, or U.S. Patent Application Publication No. 2011/0137052, the content of each of which is incorporated herein by reference.) Furan derivatives produced after hydrolyzing at least one of an ether or an ester bond could be used as chemicals or fuels, depending on remaining functionality. FIG. 2 presents a schematic process according to the present invention with the generation of an alcohol or acid.

The following examples are provided as illustration of the inventions of the present disclosure, with the recognition that altering parameters and conditions, for example by change of temperature, time and reagent amounts, and particular starting species and catalysts and amounts thereof, can affect and extend the full practice of the invention beyond the limits of the examples presented.

### 5-Butoxymethylfurfural (BMF)

Furan derivatives were synthesized from a representative alkoxymethylfurfural ether, 5-butoxymethylfurfural (BMF, 1a), using catalyst systems. Crude BMF was distilled under vacuum to obtain a furan starting material comprising 84% (BMF) and 16% butyl levulinate.

### Standard Conditions

Reaction mixtures were prepared as described below and introduced into at least one of a 100 mL MC Series Stirred Reactor (Pressure Products Industries, Warminster, Pa.) or a Multi-reactor system (Parr Industries, Moline, Ill.) with solvent and multifunctional catalyst system added. The vessel was purged with hydrogen (4×500 psi) with stirring (625 rpm). The vessel was then pressurized and heated to the desired temperature with continual stirring. After a set time, the reaction was allowed to cool to room temperature, the remaining hydrogen gas was removed, the vessel was returned to ambient pressure, the vessel was opened, the contents removed and the catalyst collected by vacuum filtration.

Initial BMF conversions were quantified by GC/MS and 1H NMR. The product selectivity (%) of products obtained using a catalyst system comprising Pd/C and Amberlyst™ alkaline resin was determined by GC-MS analysis using known standards and an internal normalization method. The product selectivity (%) of each furan derivative product is reported as the area % in the GC-MS traces. The molar yield (%) of each furan derivative is calculated as the (moles of furan derivative/moles of alkoxymethylfurfural in the starting material)*100.

### Examples 1-5

The selectivity of the hydrogenation was controlled by changing the reaction conditions. For examples 1-5, an initial series of reactions was performed in EtOAc solvent using a 5% Pd/C catalyst (0.1 g per g of BMF), at 25° C. for 60 min under 6 bar of hydrogen. The reactions were conducted with 0.3 g loadings (in relation to 1 g of BMF) of alkaline resin promoter alongside a control run carried out without the promoter. The results are provided in Table 2 below.

The furan starting material, BMF, was converted directly into the fully reduced furan derivative (7a) with 98% selectivity in EtOAc by a multifunctional catalyst system comprising 5% Pd/C with the alkaline resin promoter, Reilly™ 425 (Table 2, entry 2). In the absence of the promoter (Table 2, entry 1), only 69% selectivity of the fully reduced furan derivative was obtained. Under the same conditions using a different alkaline resin promoter, Purolite™ D5149, the selectivity to the fully reduced furan derivative decreased to 53% with partial conversion of BMF (Table 2, entry 3). An experiment was then carried out under the same conditions as described in entry 3, except that the solvent 1,4-dioxane was employed. The compositional analysis shows that 1,4-dioxane provided higher selectivity of the fully reduced furan derivative (7a) as compared to the EtOAc solvent (Table 2, entry 4). In the presence of 0.2 g triethylamine promoter and 1.0 g of 5% Pd/C (per g of BMF), BMF was converted with 20% selectivity to the partially reduced BMF (2a) and 78% selectivity to 7a (Table 2, entry 5).

TABLE 2
Effect of alkaline promoters on BMF conversion.

| | | | Product selectivity (%) | | | | | | | |
|---|---|---|---|---|---|---|---|---|---|---|
| | | | | | | | Methyl | Hydroxy- | | |
| | | | | | MTHF | | furfuryl | methyl | | |
| | | | | | alcohol | | alcohol | THF | | partially |
| | | | | Dimethyl | butyl | MTHF | butyl | butyl | | reduced |
| | | promoter | DMTHF | furan | ether | alcohol | ether | ether | BMF | BMF |
| | Promoter | (g) | (6) | (5) | (4a) | (11) | (3a) | (7a) | (1a) | (2a) |
| 1 | — | — | 1 | 3 | 4 | 1 | 0 | 69 | 0 | 0 |
| | Reilly | | | | | | | | | |
| 2 | 425 | 0.3 | 0 | 0 | 1 | 0 | 1 | 98 | 0 | 0 |
| 3 | Purolite | 0.3 | 0 | 0 | 1 | 0 | 0 | 53 | 12 | 30 |
| | D5149 | | | | | | | | | |
| 4 | Purolite | 0.3 | 3 | 0 | 2 | 1 | 0 | 92 | 0 | 0 |
| | D5149 | | | | | | | | | |
| 5 | Triethyl | 0.2 | 0 | 0 | 0 | 0 | 0 | 78 | 0 | 20 |
| | amine | | | | | | | | | |

Entry 4 is performed in 1,4-dioxane.

### Examples 6-8

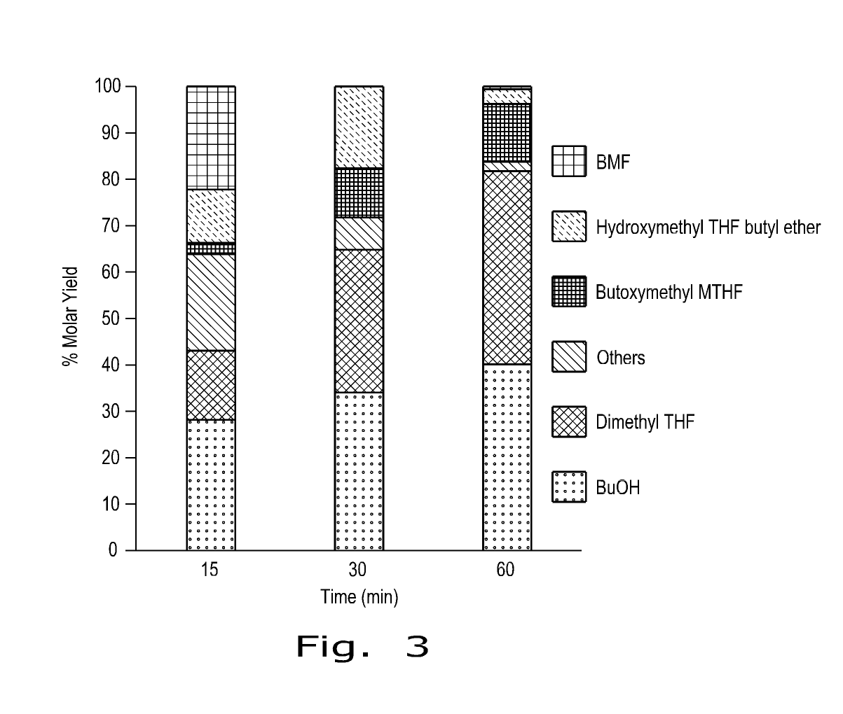

Three studies were conducted of the effect of reaction time on the hydrogenation and hydrolysis of BMF in EtOAc using a catalyst system comprising the Amberlyst™ CH28 bifunctional resin (1 g per g BMF) at the same 60° C. reaction temperatures and 60 bar hydrogen pressures. The results are shown in FIG. 3 for % molar yield and demonstrate that longer reaction times provide higher butanol recovery and dimethyltetrahydrofuran formation overall as compared to the shorter reaction time.

### Examples 9-11

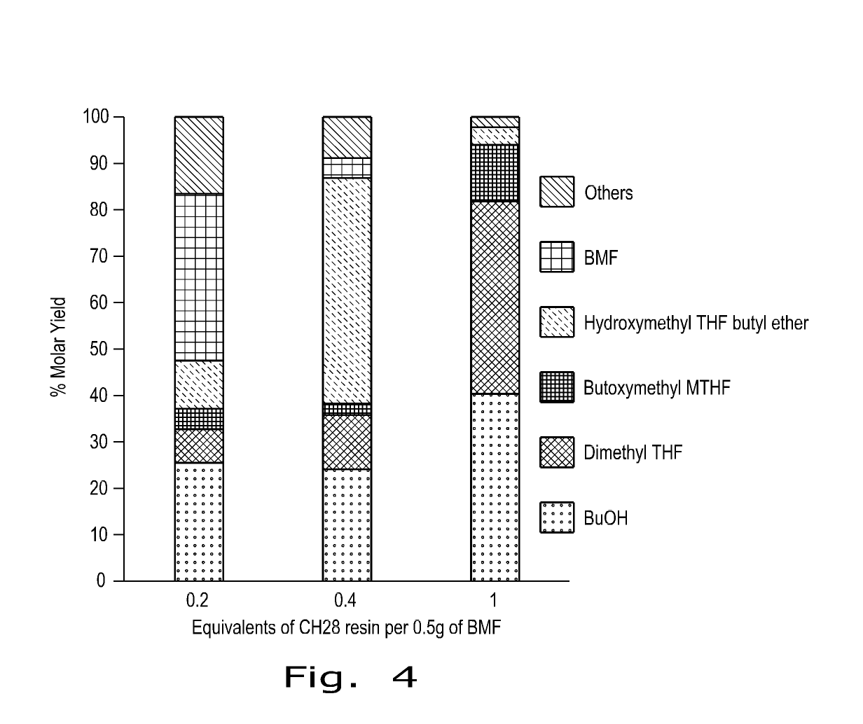

Further experiments were conducted using the same 75 mL Parr reactor arrangement, to determine the effect of equivalents of bifunctional catalyst, Amberlyst™ CH28 resin, per 0.5 g of BMF on BMF conversion, butanol recovery, and dimethyltetrahydrofuran formation. A solution of BMF in EtOAc was heated to 60° C. for 60 min with 60 bar hydrogen using various loadings of a catalyst system comprising Amberlyst™ CH28 bifunctional resin. The molar yields of furan derivatives and butanol are shown in FIG. 4. Nearly quantitative conversion of BMF was achieved using 0.4 equivalents of the bifunctional resin with the fully reduced BMF derivative the major furan derivative product and 25% BuOH recovery. The addition of higher equivalents of bifunctional resin catalyst system gave quantitative conversion of BMF, with higher recovery of butanol and formation of dimethyl THF.

### Examples 12-16

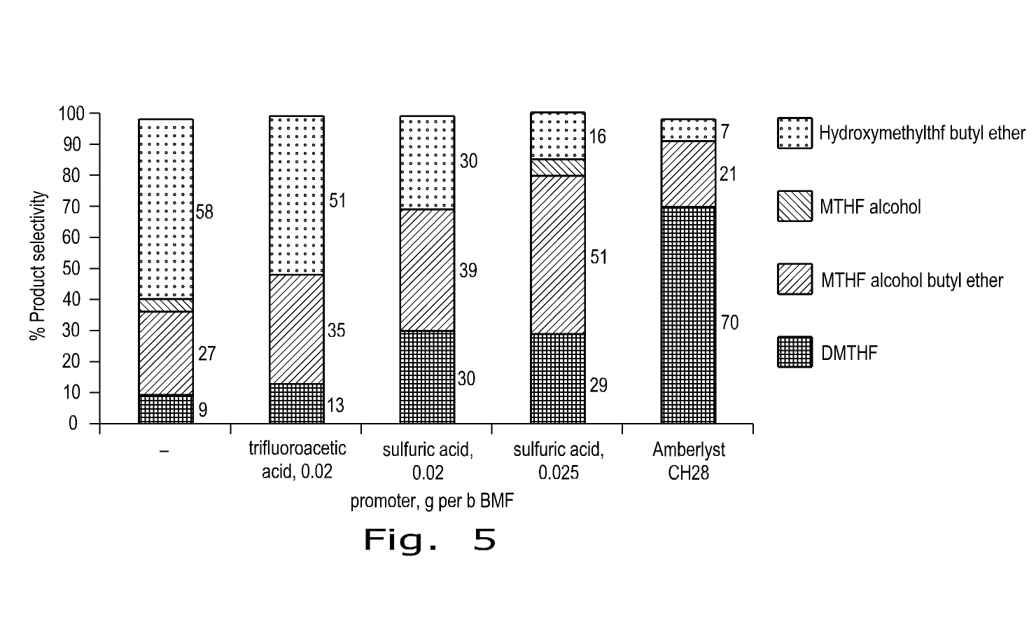

The multi-reactor system was used with a series of reactions using 10% Pd/C (0.1 g per g BMF) in EtOAc and BMF, heating at 60° C. for 60 min under 60 bar hydrogen with the promoter varied. The product selectivity produced of the various materials found in the furan derivatives product mixtures were as shown in FIG. 5. The reduced and hydrolyzed furan derivative, DMTHF, was produced at 30% selectivity in the presence of 0.02 g of sulfuric acid promoter per g of BMF, compared to only about 9% in the absence of a promoter. Surprisingly, the hydrogenation of BMF using a palladium doped acid resin catalyst system, Amberlyst™ CH28, predominantly provided 70% product selectivity for dimethyl THF. Complete hydrogenation of the furan and hydrolysis of the ether bond was obtained under mild reaction conditions.

### Examples 17-20

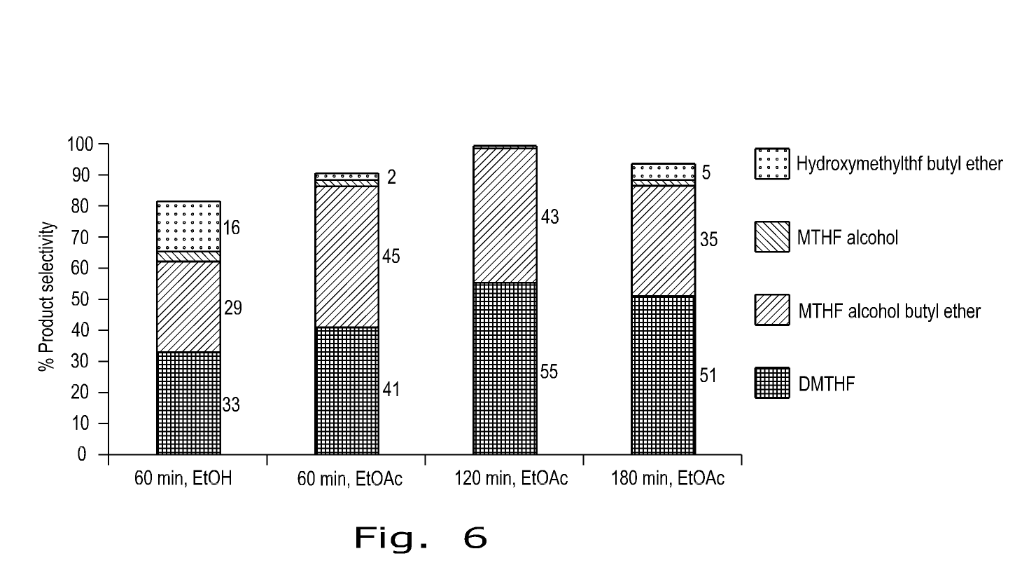

Experiments were conducted at 60° C., with 30 bar hydrogen and a multifunctional catalyst system comprising 10% Pd/C catalyst loading of 0.5 g per g BMF in the presence of H2SO4 promoter (0.08 g per g BMF). As may be observed in FIG. 6, by changing the solvent from EtOH to EtOAc, the selectivity for the furan derivative DMTHF (6) rose from 33% to 41% and the selectivity for MTHF alcohol butyl ether (4a) rose from 29% to 45%. A longer reaction time of 120 min in EtOAc solvent provided an even higher 55% product selectivity for the furan derivative DMTHF. The furan derivative MTHF alcohol butyl ether (4) was generated at 43% selectivity.

### Examples 21-32

### Recovery of Butanol for Solvent Recycle

Butanol was recovered from furan derivative product mixtures for recycling (Table 3). The highest amounts of butanol recovered were obtained from reactions performed in the presence of the catalyst system comprising the bifunctional resin, Amberlyst™ 28 (Table 3, entries 22, 24, and 29). The highest molar yield of recovered butanol was 55%.

TABLE 3
Butanol recovery from furan derivative reaction mixtures (% molar yield).

| | | Catalyst System | | | | | BuOH |
|---|---|---|---|---|---|---|---|
| | | | Amount of | Promoter, | Pressure | Time | (% molar |
| Entry # | Solvent | Catalyst | Catalyst^{a} | μL | (bar) | (hr) | yield) |
| 21 | EtOAc | CH10 | 1.0 | — | 60 | 1 | 32.9 |
| 22 | EtOAc | CH28 | 1.0 | — | 60 | 1 | 55.1 |
| 23 | EtOAc | CH28 | 1.0 | — | 60 | 0.25 | 47.3 |
| 24 | EtOAc | CH28 | 1.0 | — | 60 | 0.5 | 50.9 |
| 25 | EtOAc | 10% | 0.1 | — | 60 | 1 | 25.6 |
| | | Pd/C | | | | | |
| 26 | EtOAc | 10% | 0.1 | TFA, 13.4 | 58 | 2 | 30.6 |
| | | Pd/C | | | | | |
| 27 | EtOAc | 10% | 0.1 | H2SO4, 6.7 | 57 | 2 | 29.6 |
| | | Pd/C | | | | | |
| 28 | EtOAc | CH28 | 0.6 | — | 53 | 1 | 48.1 |
| 29 | EtOAc | CH28 | 0.8 | — | 53 | 1 | 53.1 |
| 30 | EtOAc | 10% | 0.1 | H2SO4, 5.8 | 50 | 1 | 34.2 |
| | | Pd/C | | | | | |
| 31 | EtOH | CH28 | 1.0 | — | 35 | 1 | 31.8 |
| 32 | EtOH | 10% | 0.5 | H2SO4, 23 | 23 | 1 | 47.7 |
| | | Pd/C | | | | | |

^{a}based on 1 g of BMF.

### 5-Acetoxymethylfurfural (AcMF)

Furan derivatives were synthesized from a representative acyloxymethylfurfural ester, 5-acetoxymethylfurfural (AcMF, 1b), using multifunctional catalyst systems under several conditions. AcMF was a commercial product obtained from Sigma-Aldrich.

### Examples 33-36

Using the same apparatus and standard conditions as used in examples 1-32, a series of experiments was performed using a solution of AcMF furan starting material in solvent and catalyst systems. These studies were conducted to determine the effect of varying catalyst systems, reaction times and pressures, on AcMF conversion and furan derivative selectivity. An EtOAc solution of AcMF in the presence of a catalyst system was pressurized to 60 bar hydrogen and heated for 1 h. Results indicate the catalyst system comprising a combination of Pd/C and acid promoter trifluoracetic acid (Table 4, entry 33) gave a mixture of 46% fully reduced furan derivative (7b), 26% of 4b, 16% MTHF (15), and 5% DMTHF (6). The selectivity of DMTHF under the same conditions using the multifunctional catalyst system, Amberlyst 28, gave 48% higher selectivity as compared to the combined catalyst system. Increasing the reaction time to 2 h provided even higher selectivity of DMTHF at 63%. The multifunctional catalyst system of Amberlyst 10 at 14 bar hydrogen pressure, with heating to 60° C. for 1 h, gave a 25% selectivity of the furan derivative MTHF, and partial conversion of AcMF. The results demonstrate the selectivity of furan derivatives was controlled by the choice of catalyst system, reaction temperature, and reaction time.

TABLE 4
Furan derivative selectivity from AcMF using a combination of catalyst
and acid promoter and multifunctional catalyst systems.

| | | | | Selectivity (%) | | | | | |
|---|---|---|---|---|---|---|---|---|---|
| | | | | 5- | | | 5- | | |
| | | | | methyl- | | | methyltetra- | | |
| | | | | acetoxy- | | Fully | hydrofur- | | |
| | | | | methyl | 2,5- | reduced | furyl | | |
| | Catalyst | Pressure | Time | THF | dimethyl | AcMF | alcohol | MTHF | AcMF |
| Entry | System | (bar) | (hr) | (4b) | THF (6) | (7b) | (11) | (15) | (1b) |
| 33 | 10% | 60 | 1 | 26 | 5 | 49 | 3 | 16 | 0 |
| | Pd/C, | | | | | | | | |
| | THF | | | | | | | | |
| | promoter | | | | | | | | |
| 34 | Amberlyst 28 | 60 | 1 | 9 | 53 | 13 | 16 | 1 | 1 |
| 35 | Amberlyst 28 | 60 | 2 | 10 | 63 | 9 | 15 | 0 | 0 |
| 36 | Amberylst 10 | 14 | 1 | 12 | 11 | 6 | 6 | 25 | 36 |

### Examples 37-40

The multi-reactor system was used with a series of reactions using multifunctional catalyst systems comprising either 5% Pd/C or 10% Pd/C (0.1 g per g AcMF) in either EtOAc of 1,4-dioxane and AcMF with Purolite™ D-5149 (0.3 g per g of AcMF), 7 bar hydrogen pressure and temperatures varied. The product selectivities were significantly affected by varying multifunctional catalyst systems, reaction times, and temperatures (Table 5). The highest selectivity of the fully reduced derivative of AcMF (7b) was 94% using 5% Pd/C with heating to 60° C. for 1 hour. When the reaction was performed at ambient temperature for 2 hours, the selectivity of the fully reduced derivative decreased to 86% and the selectivity of 2-methyltetrahydrofuran (15) increased to 10%. As shown in Table 5, entries 39 and 40, when the reaction was conducted under similar conditions employing either 5% or 10% Pd/C loading in either EtOAc or 1,4-dioxane, the fully reduced derivative (7b) was the primary product. The results from these experiments, in combination with results from examples 1-5, indicate that alkaline promoters increased the selectivity of the fully reduced derivatives.

TABLE 5
Effect of alkaline promoters on AcMF conversion.

| | | | | | | | 5- | |
|---|---|---|---|---|---|---|---|---|
| | | | | | | | methyltetra- | |
| | | | | 5-methyl-2- | 2,5- | Fully | hydro- | |
| | | | | acetoxymethyl | dimethyl | reduced | furfuryl | |
| | | Temp | Time | THF | THF | AcMF | alcohol | AcMF |
| Entry # | Catalyst^{a} | (C.) | (h) | (4b) | (6) | (7b) | (5) | (1b) |
| 37 | 5% Pd/C | 60 | 1 | 0 | 0 | 94 | 0 | 0 |
| 38 | 5% Pd/C | 25 | 2 | 1 | 0 | 86 | 10 | 0 |
| 39 | 10% Pd/C | 25 | 1 | 13 | 9 | 54 | 0 | 16 |
| 40 | 5% Pd/C | 25 | 1 | 0 | 0 | 49 | 0 | 50 |

^{a}Promoter is Purolite ™ D-5149 resin (0.3 g per g of AcMF) at 7 bar, Pd/C catalyst (0.1 g per g AcMF). Experiments were performed in EtOAc except entry 39.

Although the examples presented herein describe a multifunctional catalyst system that employs Pd catalysts, various other catalyst systems, such as nickel, platinum, ruthenium, or copper can also be used and may include an alkaline or acidic promoter.

### Abbreviations

Many of the chemicals and derivatives discussed in the foregoing specification are typically referred to by an abbreviation, rather than their chemical name. The most frequently used abbreviations include the following:
AcMF=5-acetoxymethyl furfural
BMF=5-butoxymethylfurfural
DMTHF=2,5-dimethyl tetrahydrofuran
EtOAc=ethyl acetate
EtOH=ethanol
HMF=5-hydroxymethylfurfural
MTHF=5-methyl tetrahydrofuran
TFA=trifluoroacetic acid

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein.

What is claimed is:

1. A method of synthesizing furan derivatives comprising, reacting a starting material comprising at least one of an alkoxymethylfurfural ester and/or an alkoxymethylfurfural ether with hydrogen in the presence of a multifunctional catalyst system under mild conditions comprising at least one of an operational temperature of up to about 150° C. and/or a pressure of up to about 1,230 psi (˜85 bar);
wherein a reduced alkyl furan derivative is formed.

2. The method according to claim 1, wherein hydrogenation of said starting material comprises hydrolyzing an ester or an ether bond, respectively, from said alkoxymethylfurfural ester or ether.

3. The method according to claim 1, further comprising recovering at least one of an acid or an alcohol as a reaction product.

4. The method according to claim 1, wherein said operational temperature is in a range from about 18° C. to about 130° C.

5. The method according to claim 4, wherein said pressure is within a range from 290 psi (˜20 bar) to about 1,015 psi (˜70 bar).

6. The method according to claim 1, wherein hydrogenation reduces said starting material in a controlled manner to selectively yield a partially or a fully reduced furan derivative.

7. The method according to claim 1, wherein said reduced alkyl furan derivative comprises at least one of: 2,5-dimethyl-tetrahydrofuran (DMTHF), (5-(butoxymethyl)furan-2-yl)methanol, 5-methyl-2-butoxymethylfuran, 5-methyl-2-butoxymethyltetrahydrofuran, 2,5-dimethylfuran, (5-(butoxymethyl)tetrahydrofuran-2-yl)methanol, 2-(butoxymethyl)-5-(ethoxymethyl)furan, 2-(butoxymethyl)-5-(ethoxymethyl)tetrahydrofuran, (5-methylfuran-2-yl)methanol, (5-methyltetrahydrofuran-2-yl)methanol, 2-(butoxymethyl)furan, 2-(butoxymethyl)tetrahydrofuran, 2-methylfuran, 2-methyltetrahydrofuran, (5-formylfuran-2-yl)methyl acetate, (5-methyltetrahydrofuran-2-yl)methyl acetate, (5-(hydroxymethyl)tetrahydrofuran-2-yl)methyl acetate, (tetrahydrofuran-2-yl)methyl acetate, (5-(hydroxymethyl)furan-2-yl)methyl acetate.

8. The method according to claim 3, wherein said method further includes recycling at least one of the recovered acid and the recovered alcohol in a second reaction operable to form at least one of an alkoxymethylfurfural ether or an acyloxymethylfurfural ester.

9. The method according to claim 1, wherein said multifunctional catalyst system includes at least one of: a bifunctional catalyst; a multifunctional catalyst system having an alkaline and hydrogenation functionality; a multifunctional catalyst system having a plurality of functionalities selected from the group consisting of acid, base, hydrogenation, dehydration, and ring opening functionalities.

10. The method according to claim 1, wherein the multifunctional catalyst system includes a heterogeneous catalyst.

11. The method according to claim 1, wherein said multifunctional catalyst system includes a catalyst that exhibits at least one of a degree of selectivity for at least one of an alkoxymethylfurfural ester and/or an alkoxymethylfurfural ether:
a) wherein the starting material comprising at least one of an alkoxymethylfurfural ester and/or an alkoxymethylfurfural ether is used to form 2,5-dimethyl-tetrahydrofuran of at least about 30% product selectivity;
b) wherein the starting material 5-butoxymethylfurfural is used to form (5-(butoxymethyl)tetrahydrofuran-2-yl)methanol of at least about 80% product selectivity;
c) wherein the starting material 5-butoxymethylfurfural is used to form (5-(butoxymethyl)furan-2-yl)methanol of at least about 30% product selectivity;
d) wherein the starting material 5-butoxymethylfurfural is used to form 2-(butoxymethyl)-5-methyltetrahydrofuran of at least about 30% product selectivity;
e) wherein the starting material 5-acetoxymethylfurfural is used to form (5-(hydroxymethyl)tetrahydrofuran-2-yl)methyl acetate of at least about 85% product selectivity;
f) wherein the starting material comprising at least one of an alkoxymethylfurfural ester and/or an alkoxymethylfurfural ether is used to form 5-methyltetrahydrofuran of at least about 25% product selectivity.

12. The method according to claim 1, wherein said multifunctional catalyst system comprises a transition metal of group VIII to XI on substrate supports.

13. The method of claim 12, wherein the multifunctional catalyst system comprises a transition metal that is at least one member selected from the group consisting of:
platinum, palladium, ruthenium, rhodium, iridium, nickel, cobalt, iron, copper, silver, and gold.

14. The method according to claim 1, wherein said multifunctional catalyst system is a Pd/C catalyst with 0.3-10% Pd loading.

15. The method according to claim 1, wherein said multifunctional catalyst system includes an alkaline promoter that is at least one of triethylamine, and/or an ion exchange resin of a polymer of unmodified 4-vinylpyridine residues and divinylbenzene residues.

16. The method according to claim 1, wherein said multifunctional catalyst systems includes an acidic promoter that is at least one of a homogeneous acid, heterogeneous acid, a mineral acid, and/or an organic acid.

17. A method of preparing furan derivatives comprising:
reacting a starting material comprising at least one of an alkoxymethylfurfural ester or an alkoxymethylfurfural ether with hydrogen in the presence of a multifunctional catalyst system hydrolyzing one of at least an ester or an ether bond, respectively, from said alkoxymethylfurfural ester or alkoxymethylfurfural ester; and
recovering at least one of an acid or alcohol and a reduced alkyl furan derivatives reaction products.

18. The method according to claim 16, wherein said alkoxymethylfurfural ester or alkoxymethylfurfural ether is reduced in a controlled manner to form a partially or fully reduced furan derivative.

19. The method according to claim 15, wherein reacting of said starting material with hydrogen is performed at least one of an operational temperature of less than 100° C., and a pressure of less than 950 psi.

20. The method according to claim 15, wherein said multifunctional catalyst system includes a catalyst that exhibits at least one of a degree of selectivity for at least one of an alkoxymethylfurfural ester and/or an alkoxymethylfurfural ether:
a) wherein the starting material comprising at least one of an alkoxymethylfurfural ester and/or an alkoxymethylfurfural ether is used to form 2,5-dimethyl-tetrahydrofuran of at least about 30% product selectivity;
b) wherein the starting material 5-butoxymethylfurfural is used to form (5-(butoxymethyl)tetrahydrofuran-2-yl)methanol of at least about 80% product selectivity;
c) wherein the starting material 5-butoxymethylfurfural is used to form (5-(butoxymethyl)furan-2-yl)methanol of at least about 30% product selectivity;
d) wherein the starting material 5-butoxymethylfurfural is used to form 2-(butoxymethyl)-5-methyltetrahydrofuran of at least about 30% product selectivity;
e) wherein the starting material 5-acetoxymethylfurfural is used to form (5-(hydroxymethyl)tetrahydrofuran-2-yl)methyl acetate of at least about 85% product selectivity;
f) wherein the starting material comprising at least one of an alkoxymethylfurfural ester and/or an alkoxymethylfurfural ether is used to form 5-methyltetrahydrofuran of at least about 25% product selectivity.

21. A method of synthesizing furan derivatives comprising, reacting an alkoxymethylfurfural ester or alkoxymethylfurfural ether with hydrogen at a temperature up to about 150° C. and pressure of up to about 85 bar for a time sufficient to form at least one of a partially and a fully reduced derivative of said alkoxymethylfurfural ester or alkoxymethylfurfural ether.

22. The method according to claim 19, wherein the method includes hydrolyzing at least one of an ester or an ether bond, respectively, from said alkoxymethylfurfural ester or alkoxymethylfurfural ester.

23. The method according to claim 19, wherein said furan derivative includes at least one of (5-(butoxymethyl)tetrahydrofuran-2-yl)methanol, (5-(butoxymethyl)furan-2-yl) methanol, 2-(butoxymethyl)-5-methyltetrahydrofuran, (5-(hydroxymethyl)tetrahydrofuran-2-yl)methyl acetate, 5-methyltetrahydrofuran, or 2,5-dimethyl-tetrahydrofuran (DMTHF).
